(19) United States
(12) Patent Application Publication
(10) Pub. No.: US 2020/0246792 A1
(43) Pub. Date: Aug. 6, 2020

(54) SINGLE UNIT ASSAY DEVICE, METHOD, AND ASSEMBLY

(71) Applicant: Charm Sciences, Inc., Lawrence, MA (US)

(72) Inventors: Steve Saul, Lawrence, MA (US); John Jabour, Lawrence, MA (US)

(73) Assignee: Charm Sciences, Inc., Lawrence, MA (US)

(21) Appl. No.: 16/652,175

(22) PCT Filed: Sep. 28, 2018
(86) PCT No.: PCT/US18/53333

§ 371 Date: Mar. 30, 2020

Related U.S. Application Data
(60) Provisional application No. 62/564,449, filed on Sep. 28, 2017.

(51) Int. Cl. B01L 3/00; G01N 33/552

(52) U.S. Cl. CPC B01L 3/5023; G01N 33/552; G01N 2333/38; B01L 2400/0406; B01L 2300/0825

(57) ABSTRACT

A single unit assay device, method, and assembly is shown and described. In one embodiment, a self-contained assembly and method analyze a sample for a presence of one or more analytes, residues, and may include comparing visual intensity of a detectable signal of a test area to a visual intensity of a control area. The result is an improved field test for efficient and effective qualitative analysis.

### REFERENCE TO PRIOR APPLICATIONS

This application claims the benefit of PCT Application No. 18/53333, filed Sep. 28, 2018, which claims be benefit of U.S. Provisional Application No. 62/564,449, filed Sep. 28, 2017, and are hereby incorporated by reference in their entireties.

### FIELD OF THE TECHNOLOGY

The present disclosure relates generally to the detection of an analyte or a residue, and more particularly to improved field test devices, methods, and assemblies.

### BACKGROUND

Various types of testing apparatuses detect the presence of one or more analytes in a sample. Onsite testing tools may be preferable for certain tasks, such as detecting contaminants in food supplies at a farm and the like. However, conventional systems and methods limit onsite applicability. For instance, current screening applications fail to provide rapid analysis without additional equipment, expertise, and/or tedious preparation.

Therefore, Applicant desires systems and methods for detecting an analyte, or a residue, effectively and efficiently without the drawbacks presented by traditional systems and methods.

### SUMMARY

In accordance with the present disclosure, test strips and systems are provided for the analysis of a sample. This disclosure provides improved test strip and tube devices and methods that are convenient, efficient, and safe for the user, particularly when used to detect the presence or absence of an analyte in a sample free of additional equipment and/or expertise.

One embodiment of the present disclosure includes a method for analyzing a grain sample for a presence of one or more analytes including providing a sample tube having an extraction material and a test strip with a glass fiber membrane supporting a control area and a test area; removing the test strip from a releasable cap of the sample tube; adding a predetermined volume of the grain sample into the sample tube; adding a predetermined volume of water into the sample tube; solubilizing the extraction material, the grain sample, and the water to define a solution adapted to extract the analyte, when present; introducing the test strip into the solution; incubating the tube free of an incubator; and comparing intensity of a detectable signal of the test area to the control area, wherein a greater intensity of the detectable signal in the test area as compared to the control area indicates a negative result for a particular analyte and a greater intensity of the detectable signal in the control area compared to the test area indicates a positive result for the particular analyte.

In some examples, the method includes mixing the solution by manipulating the sample tube prior to introducing the test strip. The method may include mixing the solution including manipulating the sample tube free of a centrifuge. The method may include providing the extraction material includes providing an extraction material housed within the sample tube. The method may include providing a Fusion 5 membrane substrate adhered to a solid support on the test strip. The method may include a Fusion 5 membrane substrate that maintains adhesion to the test strip during operation.

In certain examples, the method may include adding a predetermined volume of the grain sample includes measuring a capful volume of a measuring removable cap. The method may include adding water includes measuring two capfuls volume of water. The method may include adding water free of a pipette. The method may include comparing intensity of the detectable results directly on the test strip without equipment.

Another embodiment of the disclosure is a single unit assay for the analysis of a sample having a sample tube having a releasable cap; an extraction material housed within the sample tube; and a test strip removably housed within the sample tube and comprising: a solid backing support; and a glass fiber membrane adhered to the solid backing support and including at least one control zone and at least one test zone.

In some examples, the glass fiber membrane comprises a Fusion 5 membrane substrate. The Fusion 5 membrane may maintain adhesion about the solid backing support following a fluid submersion within the sample tube. The test strip may include two or more control zones and two or more test zones for multiple analytes. The test strip may comprise an aflatoxin test strip or the like.

In certain examples, the extraction material comprises at least one extraction material. The sample tube having a removable cap to deliver a predetermined volume of sample to the tube. For instance, the removable cap may deliver one capful, or the like, of a grain sample to the tube. Further, the sample tube having a removable cap may deliver a predetermined volume of solution to the tube. For instance, the removable cap may deliver two capfuls, or the like, of water, or any equivalent, to the tube.

The above summary was intended to summarize certain embodiments of the present disclosure. Embodiments will be set forth in more detail in the figures and description of embodiments below. It will be apparent, however, that the description of embodiments is not intended to limit the present inventions, the scope of which should be properly determined by the appended claims.

### BRIEF DESCRIPTION OF THE DRAWINGS

Embodiments of the disclosure will be better understood by a reading of the Description of Embodiments along with a review of the drawings, in which:

FIG. 1 is an exploded view of a single unit assay according to an embodiment of the disclosure;

FIG. 2 is a side perspective view of one embodiment of an isolated test strip according to FIG. 1;

FIG. 3 is a top view of one embodiment of an isolated test strip according to FIG. 1; and

FIG. 4 is a front perspective view of one embodiment of completion of a test assembly according to FIG. 1.

### DESCRIPTION OF EMBODIMENTS

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

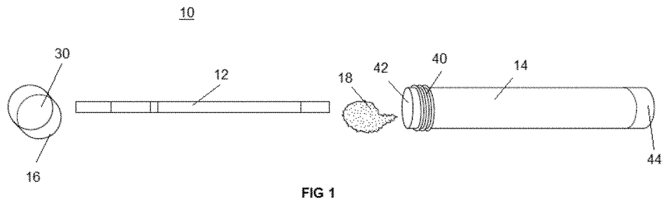

Referring now to the drawings in general, it will be understood that the illustrations are for the purpose of describing embodiments of the disclosure and are not intended to limit the disclosure or any invention thereto. As best seen in FIG. 1, one embodiment of a single unit assay 10 includes a test strip 12, sample tube 14, extraction material 18, and a removable cap 16 for qualitative analyte, reside, or the like screening. In certain embodiments, the qualitative screening includes visual interpretation of intensities on the test strip 12 after completion of an equipment-free testing procedure.

Those skilled in the art having the benefit of this disclosure will recognize a variety of self-contained unit configurations and applications. As shown in FIG. 1, the sample tube 14 includes a closed distal portion 44 with an opposing open proximate portion 42 adapted to provide access, i.e. delivery of any of the elements shown and described herein, to the tube 14. The removable cap 16 may be removably secured about the tube 14 in a variety of configurations, including a threaded orientation 40 having an open mating end 30, as illustrated in FIG. 1.

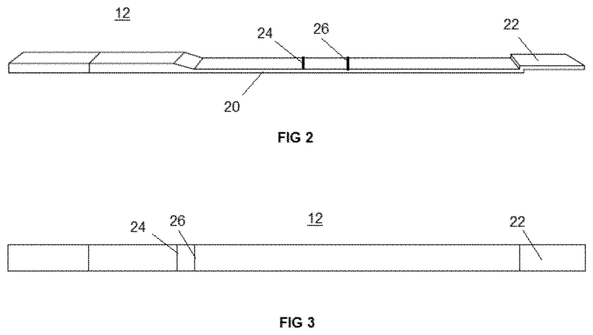

Similarly, those skilled in the art having the benefit of this disclosure will recognize a variety of test strip applications to match the detection of a particular analyte and/or residue. For instance, any of the elements and teachings of U.S. Pat. Nos. 5,985,675, 6,319,446, 6,475,805, 7,097,983, 7,410,808, 7,785,899, 7,785,899, 7,897,365, 8,481,334, 8,481,334, 8,592,171, 8,592,171, and 9,057,724 as well as U.S. application Ser. No. 14/372,088 may be useful for the inventions shown and described herein, and are therefore incorporated by reference where consistent and useful as understood by those skilled in the art. In addition, as shown in FIG. 2, test strip 12 includes a solid support 20 with a membrane adhered to at least one side of the solid support 20. Test strip 12 may provide any combination of test zones/areas/lines shown and described herein, and FIGS. 2 and 3 illustrate one example of control zone 24 and a test zone 26.

The strip can also be wholly or partially of a material to bind proteins, such as carrier proteins for example, an extraction material or the like. A variety of materials can be used in various portions of the strip including fiberglass or glass fiber filter 22, for example WHATMAN Fusion 5 membrane (Whatman is a registered trademark of Whatman paper Limited, Kent, England). Solid support 20 provides a structural foundation for test strip 12 wherein any of various strip components shown and described herein may be attached. Solid support 20 may be comprised of any combination of plastics, such as polystyrene. In particular examples, a cover layer is aligned along the upper portion of the nitrocellulose. The cover layer may protect the nitrocellulose from contamination. Further, the cover layer may provide a capillary barrier, for instance to push sample flow up the strip as shown and described herein, for instance when the test strip is free of a sponge. In particular examples, the cover layer is a nonporous, non-liquid permeable membrane. Further, the cover layer may include an adhesive, for instance a semi- or clear adhesive to allow visual interpretation of line/zone intensities through the layer(s).

Embodiments of the extraction material include a variety of formulations and compositions for screening of a particular analyte, reside, and the like and/or at an associated concentration level. The Applicant has unexpectantly discovered the extraction material in this qualitative visual test procedure may provide both a blocking agent, for instance for the nitrocellulose, while assisting to block binding sites to improve flow. For instance, the blocking agent may flow ahead of bead flow and block the nitrocellulose ahead of the beads at the test zone(s) and control zone(s). Example of the extraction material includes a variety of proteins usefully employed alone or in combination, including, but not limited to, bovine collagen, ovalbumin, keyhole limpet hemocyanin, and thyroglobulin, albumin, e.g., fish serum albumin, bovine serum albumin, and the like, gelatin peptone, soy peptone, soy/casein Primatone, and Primatone RL. In one example, an aflatoxin screening detection extraction material includes about sixty to about ninety-five percent serum albumin, about two to about twenty percent buffer material, and about one to about fifteen percent anionic detergent. Still a further aflatoxin screening detection extraction material includes about seventy to about ninety percent serum albumin, about three to about ten percent buffer material, and about two to about ten percent anionic detergent. While alternative embodiments include additional combinations thereof for establishing the improvements shown and described herein.

After completion of the testing procedure, a higher intensity at a test zone read visually, i.e. without a reader or the like equipment, generally indicates a negative result (i.e., absence of analyte) whereas a higher intensity at a control zone indicates a positive result (i.e., presence of analyte). In some examples, a false negative result may be caused by low sensitivity or low concentration of analyte. Similarly, a false positive result may be caused by oversensitive or unspecific binding to substances within the sample. Test sensitivity may be further adjusted to address environmental conditions, i.e. temperature, humidity, and the like, sample flow conditions, and by adding a mixture of additional receptors to the test strip.

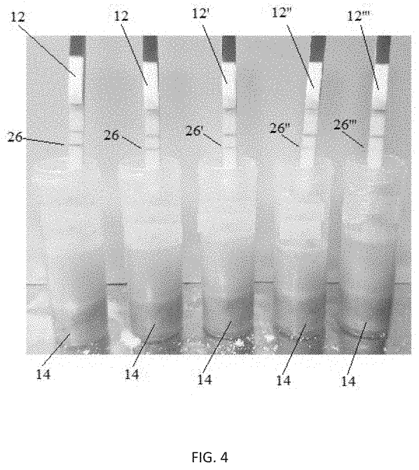

FIG. 4 illustrates one embodiment of differing test result intensities at completion of a testing operation shown and described herein. The five sample assemblies indicate visual findings, for instance field testing, of differing concentrations at the respective test zones 26 free of an incubator, reader machinery, and the like. As illustrated, the test strips 12 visually, i.e. without a reader or the like equipment, have a higher intensity at a test zones 26 to indicate a negative result (i.e., absence of analyte at a predetermined concentration). Whereas the test strips 12′,12″, 12′″ visually, i.e. without a reader or the like equipment, present a lower intensity at test zones 26′, 26′, and 26′″ to indicate a positive result (i.e., presence of analyte at a predetermined concentration). In this particular example, the predetermined screening level was twenty parts-per-billion, wherein test strips 12 visually indicate higher intensity at a test zones 26 than the screening level to indicate a negative result. Whereas the test strips 12′,12″, 12′″ visually indicate lower intensity at a test zones 26′, 26′, and 26′″ than the screening level to indicate a positive result. For example, the test zone 26′ of test strip 12′ visually indicates a test result of a twenty parts-per-billion concentration. The test zone 26″ of test strip 12″ visually indicates a test result of a thirty parts-per-billion concentration, i.e. less intensity at the test zone 26′ than the negative result intensity of test zone 26. Further, the zone 26′″ of test strip 12′″ visually indicates a test result of a one hundred parts-per-billion concentration, i.e. clearly visually indicates less intensity at the test zone 26′ than the negative result intensity of test zone 26. Those having the benefit of this disclosure will recognize a variety of visual indicator orientations and arrangements for screening differing analyte/residue concentrations as supported herein.

Numerous characteristics and advantages have been set forth in the foregoing description, together with details of structure and function. Many of the novel features are pointed out in the appended claims. The disclosure, however, is illustrative only, and changes may be made in detail, especially in matters of shape, size, and arrangement of parts, within the principle of the disclosure, to the full extent indicated by the broad general meaning of the terms in which the general claims are expressed. It is further noted that, as used in this application, the singular forms “a,” “an,” and “the” include plural referents unless expressly and unequivocally limited to one referent.

What is claimed is:

1. A single unit assay assembly for the analysis of a sample, said single unit assay assembly comprising:
a. a sample tube having a releasable cap;
b. an extraction material housed within said sample tube; and
c. a test strip removably housed within said sample tube and comprising:
i. a solid backing support, and
ii. a glass fiber membrane adhered to said solid backing support and including at least one control zone and at least one test zone, and
wherein said test strip removably housed around said extraction material in a self-contained assembly.

2. The single unit assay of claim 1, wherein said glass fiber membrane comprises a Fusion 5 membrane substrate.

3. The single unit assay of claim 2, wherein said Fusion 5 membrane adapted to maintain adhesion about said solid backing support following a fluid submersion within said sample tube.

4. The single unit assay of claim 1, wherein said test strip includes two or more control zones and two or more test zones for multiple analytes.

5. The single unit assay of claim 1, wherein a greater visual intensity of said detectable signal in said test area at test completion as compared to a visual intensity control area at test completion indicates a negative result for a particular analyte.

6. The single unit assay of claim 1, wherein a greater visual intensity of said detectable signal in said control area at test completion compared to a visual intensity of said test area at test completion indicates a positive result for said particular analyte.

7. The single unit assay of claim 1, wherein said sample tube having a removable cap adapted to deliver a predetermined volume of sample to said tube.

8. The single unit assay of claim 7, wherein said removable cap adapted to deliver one capful of a grain sample to said tube.

9. The single unit assay of claim 1, wherein said sample tube having a removable cap adapted to deliver a predetermined volume of solution to said tube.

10. The single unit assay of claim 1, wherein said single unit assay adapted to screen for an aflatoxin.

11. The single unit assay of claim 1, wherein said single unit assay assembly adapted to develop said analysis of said sample in an ambient temperature environment.

12. The single unit assay of claim 1, wherein said single unit assay assembly adapted to develop said analysis of said sample in a substantially room temperature environment.

13. The single unit assay of claim 1, wherein said single unit assay assembly adapted to develop said analysis of said sample without incubation.

14. A method for analyzing a grain sample for a presence of one or more analytes in a self-contained assembly, said method comprising:
a. providing a sample tube having an extraction material and a test strip with a glass fiber membrane supporting a control area and a test area;
b. removing said test strip from a releasable cap of said sample tube;
c. adding a predetermined volume of said grain sample into said sample tube;
d. adding a predetermined volume of water into said sample tube;
e. solubilizing said extraction material, said grain sample, and said water to define a solution adapted to extract said analyte, when present;
f. introducing said test strip into said solution;
g. incubating said tube free of an incubator; and
h. comparing visual intensity of a detectable signal of said test area to a visual intensity of said control area, wherein a greater intensity of said detectable signal in said test area as compared to said control area indicates a negative result for a particular analyte and a greater intensity of said detectable signal in said control area compared to said test area indicates a positive result for said particular analyte.

15. The method of claim 14, including mixing said solution by manipulating said sample tube prior to introducing said test strip.

16. The method of claim 14, wherein providing said extraction material includes providing an extraction material housed within a self-contained sample tube.

17. The method of claim 14, further including providing a Fusion 5 membrane substrate adhered to a solid support on said test strip.

18. The method of claim 17, wherein said Fusion 5 membrane substrate maintaining adhesion to said test strip during operation.

19. The method of claim 14, wherein adding a predetermined volume of said grain sample includes measuring a capful volume of a measuring removable cap.

20. The method of claim 14, wherein comparing intensity of said detectable signals includes visually observing said test strip without equipment.
